(19) United States
(12) Patent Application Publication
(10) Pub. No.: US 2012/0027856 A1
(43) Pub. Date: Feb. 2, 2012

(54) PHARMACEUTICAL PRODUCT COMPRISING YEAST

(75) Inventors: Bodo Asmussen, Bendorf (DE); Christiane Schiller, Stralsund (DE); Werner Weitschies, Neuenkirchen (DE); Grzegorz Garbacz, Greifswald (DE); Mahmoud A. Omer-Adam, Greifswald (DE); Stefan Nagel, Greifswald (DE)

(73) Assignee: LTS Lohmann Therapie-Systeme AG, Andernach (DE)

(21) Appl. No.: 12/733,247

(22) PCT Filed: Aug. 21, 2008
(86) PCT No.: PCT/EP2008/006897

§ 371 Date: Apr. 30, 2010

(30) Foreign Application Priority Data
Sep. 1, 2007 (DE) 10 2007 041 588.7

(51) Int. Cl. A61K 9/28; C12P 1/02; A61P 25/04; A61K 31/167; A61K 9/14

(52) U.S. Cl. 424/474; 514/630; 424/494; 424/497; 424/490; 435/171

(57) ABSTRACT

A pharmaceutical product containing an active pharmaceutical ingredient and having a controllable release of the active pharmaceutical ingredient. The pharmaceutical product comprises yeast that is capable of fermentation. The carbon dioxide production of the fermentation releases the active pharmaceutical ingredient from the pharmaceutical product.

### CROSS-REFERENCE TO RELATED APPLICATIONS

This application is a National Stage application of International Application No. PCT/EP2008/006897, filed on Aug. 21, 2008, which claims priority of German application number 10 2007 041 588.7, filed on Sep. 1, 2007, both of which are incorporated herein by reference in their entireties.

### BACKGROUND OF THE INVENTION

1. Field of the Invention

The present invention relates to a pharmaceutical product having controllable release of the active pharmaceutical ingredient it contains, which comprises yeast capable of fermentation, the carbon dioxide production of which releases the active pharmaceutical ingredient from the pharmaceutical product.

2. Description of the Prior Art

In the drug therapy of numerous diseases, for example infectious diseases, disorders of the cardiovascular system, allergies, conditions of pain or disorders of the hormone balance, it is desired to maintain a constant level of active pharmaceutical ingredient in the blood or tissue for a prolonged period of time. To achieve this, dosage forms having a modified active ingredient release are being used, especially for active pharmaceutical ingredients that do not have a long half-life in the plasma. Such dosage forms include systems having prolonged active ingredient release, systems having delayed release, and systems having a pulsatile release of active ingredient. A main aim here is to reduce the intraindividual and interindividual variability of plasma levels. In particular, the aim is to prevent plasma level peaks and thereby an increased risk of side effects. Moreover, by using these dosage forms, it is possible to avoid the incorrect taking of medication, and the compliance of patients can be improved, especially on account of the easy handling and reduced frequency of taking the medication.

Dosage forms having modified active ingredient release are also being utilised in the drug treatment of disease conditions which are subject to circadian rhythms and wherein the symptoms tend to occur more frequently or more severely at certain times of the day.

In the state of the art, numerous possibilities are known for influencing the active pharmaceutical ingredient release from a pharmaceutical product. For example, the physicochemical properties of the active pharmaceutical ingredient can be modified such that its dissolution rate is altered. This includes, inter alia, binding the active pharmaceutical ingredient to an ion exchange resin, or selecting the particle size. Pharmaco-technological measures concern the choice of excipients and the design of the dosage form. Thus, a modification of the release of active pharmaceutical ingredient can be achieved via the properties of coatings or of matrix-forming agents or the structure of the dosage form. With respect to the kinetics of the active ingredient release, diffusion-controlled, erosion-controlled and osmotically controlled release systems can be distinguished in general.

### SUMMARY OF THE PRESENT INVENTION

In the known pharmaceutical products for controlled active pharmaceutical ingredient release, the release of the active pharmaceutical ingredient is, however, not independent of the ambient conditions, so that the active ingredient release may be subject to unpredictable fluctuations. The object of the present invention was therefore to develop a pharmaceutical product wherein the active pharmaceutical ingredient release takes place as independently from ambient conditions as possible.

This object is achieved by providing a pharmaceutical product which contains microorganisms that are capable of alcoholic fermentation and which are medically acceptable, for example yeast. After administration of the pharmaceutical product, the release of the active pharmaceutical ingredient contained in the pharmaceutical product is controlled by the course of the carbon dioxide production which occurs when the alcoholic fermentation begins to take place.

Pharmaceutical products containing yeast cells are already known. For example, capsules are available under the trade names PERENTEROL® and YOMAGI® which contain the medicinal yeast Saccharomyces boulardii. In these pharmaceutical products, which are to be used against diarrhoeal diseases, the yeast itself is to be regarded as the therapeutically active component of the pharmaceutical product since its function is to regenerate the intestinal flora and thereby to counteract diarrhoea.

Furthermore, pharmaceutical products are known in the state of the art within which carbon dioxide is formed after administration of the pharmaceutical products. Thus, for example, gastroretentive systems are described in the published applications WO 03/0112 A1 and US 2006/003003 A1 which use the buoyancy produced by the formation of carbon dioxide for floating on the chyme or on the liquid contained in the stomach. In this way, a prolonged retention time of the pharmaceutical product in the stomach can be achieved.

According to WO 2006/024638 A2, the carbon dioxide being formed after administration of a pharmaceutical product can also be used for mixing the active pharmaceutical ingredient with the food in the stomach, which is to induce a retarding effect.

Carbon dioxide production within pharmaceutical products can also be utilised to accelerate the disintegration of the dosage form after its administration, as is described, for example, in JP 2003 231629 A.

In the known pharmaceutical products using the carbon dioxide being generated after their administration, the production of carbon dioxide is caused by exposing a salt, usually sodium carbonate, to an acid. This chemical reaction is difficult to control, if at all possible.

The present invention is based on considerations according to which an active pharmaceutical ingredient release that is largely independent of ambient conditions would necessitate a controllable carbon dioxide production, and according to which it might be possible to control the carbon dioxide production which occurs during alcoholic fermentation in a pharmaceutical product.

Surprisingly, examinations carried out by the applicant have shown that the kinetics of the release of an active pharmaceutical ingredient from a pharmaceutical product can indeed be controlled by carbon dioxide being formed in the fermentation of yeast.

### BRIEF DESCRIPTION OF THE DRAWINGS

FIG. 1 shows the time-dependent course of the volumetric expansion in 20 ml OMNIFIX® syringes caused by carbon dioxide formation during the fermentation of yeast.

FIG. 2 shows the time-dependent course of the release of paracetamol from a model pharmaceutical brought about by carbon dioxide formed in the fermentation of yeast.

FIG. 3 shows a schematic presentation of a monolithic, coated release system for active pharmaceutical ingredient, said system being based on sugar syrup, yeast and active pharmaceutical ingredient.

### DETAILED DESCRIPTION OF THE PRESENT INVENTION

The subject matter of the present invention is thus pharmaceutical products which, in addition to the active pharmaceutical ingredient they contain, comprise pharmaceutically acceptable yeast cells that are capable of fermentation. Within the meaning of the present invention, the yeast cells contained in the pharmaceutical products invariably are a plurality of yeast cells, even where in the following the term “yeast” is used in its singular form.

The yeasts, or blastomycetes, belong to the protoascomycetes. The type of asexual reproduction typical for yeasts, in the broadest sense, is cell budding. Some yeasts, however, reproduce by fission or by transitional forms between fission and budding. Thus, buds may remain connected to each other as agglomerates of buds or as pseudomycelia, or they may separate from each other completely, a large number of species of yeast forming non-septated or septated hyphae.

Yeasts which are especially suitable for use in the pharmaceutical products according to the invention are those employed in the baking, brewing and wine industries. The Saccharomycetes Saccharomyces cerevisiae, Saccharomyces carlsbergensis, Saccharomyces uvarum, Saccharomyces boulardii, Saccharomyces exiguus and Saccharomyces ludwigii can be mentioned as examples of such yeasts. Non-Sacchoromycetes such as Candida famala, Candida stellata, Dekkera bruxellensis, Hanensula uvarum, Kluyveromyces lactis, Kluyveromyces thermotolerans, Metschnikowia pulcherrima or Torulaspora delbrueckii may however be used as well.

Preferably, pure yeast strains are employed in the pharmaceutical product according to the invention. In a preferred embodiment, the pharmaceutical product contains baker's yeast (Saccharomyces cerevisiae). It is, however, also possible to use two or more strains of yeast in combination with each other in order to control the course of the fermentation within the pharmaceutical product.

In principle, it is possible for the pharmaceutical product to contain the yeast as fresh yeast. It is, however, especially preferred to use yeast in the form of instant dry yeast to produce the pharmaceutical products.

In alcoholic fermentation, glucose is metabolised by yeast, under anaerobic conditions, into ethanol and carbon dioxide:

C₆H₁₂O₆→2C₂H₅OH+2CO₂

Glucose is thus an essential educt for alcoholic fermentation. Specific embodiments of the pharmaceutical product according to the invention may exploit the fact that the chyme in the digestive tract contains carbohydrates and particularly glucoses which can be used for fermentation by the yeasts contained in the pharmaceutical product. With these embodiments it is not necessary for the inventive pharmaceutical product to also contain the sugar required for fermentation, provided that the sugar contained in the chyme can enter the pharmaceutical product.

In preferred embodiments, however, the pharmaceutical product according to the invention also contains the carbohydrate(s) necessary for fermentation. With particular preference, the pharmaceutical product contains glucose.

Instead of, or in addition to, glucose, other sugars, or mixtures thereof, which can be used for fermentation by the yeasts contained in the pharmaceutical product, may be used as well. For example, fructose, galactose, saccharose, maltose, maltotriose, raffinose, or any mixtures of these sugars, may be used in the pharmaceutical product. It is furthermore possible to use sugars or starch derivatives, for example dextrins.

The pharmaceutical products comprise a coating. The following are suitable as materials for the production of the coatings:
cellulose ethers, for example hydroxypropyl methyl cellulose or ethyl cellulose;
cellulose esters, for example cellulose acetate, cellulose acetate butyrate or cellulose acetate propionate;
polyacrylates and polymethacrylates, for example the products commercially available under the trademarks EUDRAGIT® RS, EUDRAGIT® RL or EUDRAGIT® NE;
polyvinyl derivatives such as, for example, polyvinyl acetate;
copolymers of polymethyl vinyl ether and malonic acid, or the ethyl esters, isopropyl esters and n-butyl esters thereof, for example the product available commercially under the trademark GANTREZ® AN.

It is also possible to use mixtures of the above mentioned polymers for the coatings, for example ethyl cellulose and hydroxypropyl cellulose in a weight ratio of 60:40.

Furthermore, it is possible to add suitable excipients to the coating by which the properties of the coatings can be modified. Suitable excipients are, for example, plasticiser(s), wetting agent(s) or pigment(s). Examples of plasticisers which may be used are esters such as triethyl citrate, tributyl citrate, acetyl triethyl citrate, dibutyl tartrate, diethyl sebacate, dimethyl phthalate, diethyl phthalate, dioctyl phthalate, castor oil, sesame seed oil, glyceryl triacetate, glyceryl diacetate, higher alcohols, for example glycerine or 1,2-propylene glycol, or polyethers, for example polyethylene glycols.

Suitable wetting agents are, for instance, PEG 400 stearate, sorbitan monooleate and PEG sorbitan monooleate.

Suitable pigments are, for instance, titanium dioxide and iron oxide.

By adding such excipients it is possible to modify the properties of the coatings since the mechanical properties thereof, such as flexibility, brittleness and strength, as well as the layer thickness of the coating, have an impact on the release of the active pharmaceutical ingredient.

Thus, in one embodiment, the pharmaceutical product according to the invention may have a structure analogous to that of an osmotically controlled release system, so that the carbon dioxide formed as a result of the fermentation will force an active ingredient solution or active ingredient suspension outwards, through a release aperture in the pharmaceutical product. In this way it is possible to realise a continuous release of active pharmaceutical ingredient that extends over a prolonged period of time.

The preferred coating for this embodiment is a coating of cellulose acetate since it is characterised by a particularly high strength.

In other embodiments, the pharmaceutical product may be a release system which releases the active pharmaceutical ingredient in a burst or in a pulsatile fashion. In these cases, the release may take place comparatively quickly or after a delay in time. To determine the release of the active pharmaceutical ingredient, the system may be monolithic or consist of a plurality of individual units (multiple units). If all of the multiple units have the same release properties, it is possible to realise a burst release of the active pharmaceutical ingredient contained in the multiple units. If the multiple units have different release properties, a pulsatile release of the active pharmaceutical ingredient may be possible.

Depending on the properties of the coating, the yeast may start fermenting immediately after the administration of the dosage form, or with a delay in time, so that pharmaceutical products according to the present invention which have a delayed release of active pharmaceutical ingredient are also possible.

The preferred coatings for release systems having a burst-like, pulsatile and/or delayed release of active pharmaceutical ingredient are coatings of ethyl cellulose or on the basis of ethyl cellulose, the properties of which can be modified by means of plasticisers and/or by changing the thickness of the layer. For example, the addition of a plasticiser, such as triethyl citrate, will increase the flexibility and strength of the coating to a greater extent than the addition of dibutyl sebacate.

Especially where dry yeast is used, water is required to effect the rehydration of the yeast. Water can be introduced into the dosage form from outside, either when taking the dosage form, or in the form of water already present in the gastrointestinal tract. In a specific embodiment, the water required for rehydration is already contained in the dosage form. To prevent a premature activation of the yeast and of its fermentation, within the dosage form, the water must initially be kept separate from the yeast by accommodating it in a separate compartment. To activate the yeast, the wall of the compartment containing the water must be destroyed, for example by crushing it. The water thereby comes into contact with the yeast and is thus able to rehydrate the yeast and trigger the fermentation.

### Example 1

To examine whether small amounts of educts already suffice to produce enough carbon dioxide to be able to realise the release of active pharmaceutical ingredient, between 25 mg and 100 mg of yeast, up to 100 mg glucose monohydrate and 500 μl purified water are mixed with each other and filled into a 20 ml disposable syringe (OMNIFIX®), the injection aperture of which was closed. After insertion of the plunger, the filled syringes were incubated at 37° C. The movement of the plunger, which indicates the volume increase in the syringe, was recorded as a measure for the production of carbon dioxide.

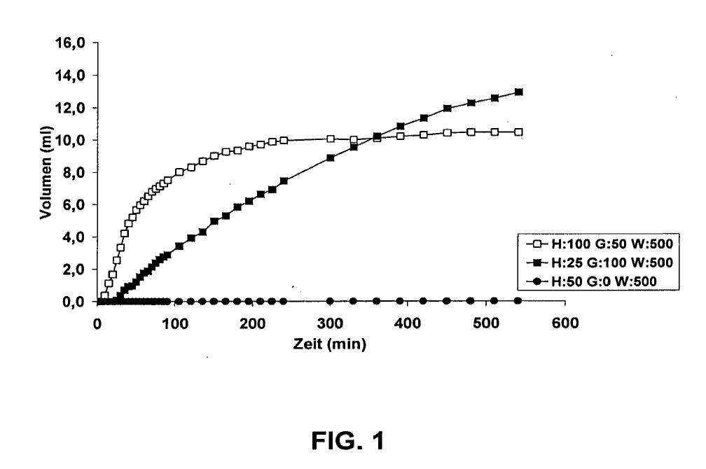

The results of this experiment are shown in FIG. 1, in which the amounts of yeast (Y) that were used are indicated in mg, the amounts of glucose monohydrate (G) used are given in mg, and the amounts of Aqua purificata (A) are given in μl values indicated are the mean values of 6 individual values. Apart from the time-dependent course of the volume expansion, it is evident that the ratio of the amounts of yeast and glucose used has an impact on the kinetics of the production of carbon dioxide. This can be exploited in the manufacture of pharmaceutical products for adjusting the release rate of the active pharmaceutical ingredient contained in the pharmaceutical product.

### Example 2

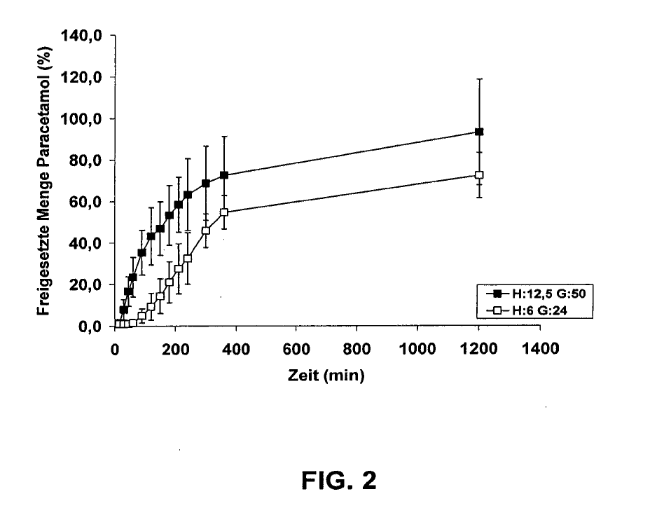

The purpose of a further-reaching experiment was to clarify whether it is possible to release an active pharmaceutical ingredient over a sufficiently long period of time by the carbon dioxide being formed during fermentation. To this end, paracetamol, in a mixture of polyethylene glycol and highly dispersed silicon dioxide, was filled into a disposable syringe. The mixture was covered with a small plate inserted in the syringe. Subsequently, a mixture of yeast, glucose and water was placed on the platelet, and the syringe was closed at the filling aperture. The respective amounts of yeast (Y) in mg, glucose monohydrate (G) in mg can be seen from the legend of FIG. 2. The amount of water used for activation was 125 μl.

The model dosage form thus created was incubated in a dissolution tester at 100 rpm and 37° C. in 900 ml water, and the amount of paracetamol that was released to the surrounding water was determined at various points in time.

The results of the above experiments are shown in FIG. 2. The measurements shown are mean values of n=3.

It was found that it is possible to release the active pharmaceutical ingredient from the model pharmaceutical product over a prolonged period of time with the aid of carbon dioxide generated by fermentation. In addition, the kinetics of the release of active pharmaceutical ingredient is dependent on the amount of yeast used. Hence, it is possible to control the release rate for the active ingredient by means of the amount of yeast and/or of glucose in the pharmaceutical product.

### Example 3

“Osmotically” controlled release system

Analogously to an osmotically controlled release system, carbon dioxide being formed can produce a pressure within the dosage form which forces a solution/suspension of active pharmaceutical ingredient outwards through a release opening. Such a release system was prepared as follows:

Active ingredient layer

| | | |
|---|---|---|
| Inner phase | Active ingredient | 22.5%-wt. |
| | Hydroxypropyl methyl cellulose | 6.0%-wt. |
| | Polyethylene oxide | 70.0%-wt. |
| Outer phase | Magnesium stearate | 1.0%-wt. |
| | highly dispersed silicon dioxide | 0.5%-wt. |

Expanding layer

| | | |
|---|---|---|
| Inner phase | Polyethylene oxide | 49.0%-wt. |
| | Glucose monohydrate | 40.0%-wt. |
| | Dry yeast | 10.0%-wt. |
| Outer phase | Magnesium stearate | 1.0%-wt. |

The components of the respective inner phases of both layers were granulated separately. Subsequently, the respective outer phase was added and both granulates were compressed into a biconvex bilayer tablet. The tablets thus obtained were provided with a water-permeable, gas-tight coating, for which coating 20 g cellulose acetate had been dissolved in 970 ml of acetone and mixed with 4.5 g of polyethylene glycol 400, which had been dissolved in 30 ml water. Thereafter, an opening for the release of active ingredient was drilled into the coating in the region of the active ingredient-containing layer of the bilayer tablet.

### Example 4

A release system having a burst release of active ingredient was prepared as follows:

Sugar syrup

| | |
|---|---|
| Glucose monohydrate | 71.4% |
| Purified water | 28.6% |

Dosage form

| | |
|---|---|
| Sugar syrup | 40% |
| Micronised dry yeast | 40% |
| Active pharmaceutical | |
| ingredient paracetamol | 20% |

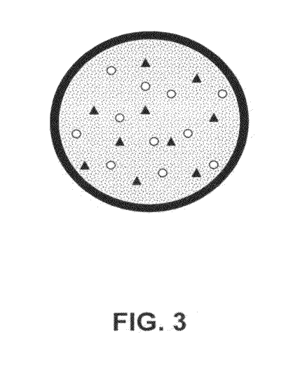

First the sugar syrup was prepared. On cooling, the dry yeast and the active pharmaceutical ingredient was incorporated in the syrup and small spheres were formed, which were then provided with a water-permeable coating that was as gas-tight as possible, for which coating 20 g ethyl cellulose had been dissolved, together with 4 g dibutyl sebacate, in 200 ml of ethanol. This active pharmaceutical ingredient release system comprising yeast (▴) and active pharmaceutical ingredient (∘) is shown in FIG. 3.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A pharmaceutical product containing an active pharmaceutical ingredient for the controlled release of said active pharmaceutical ingredient, wherein said pharmaceutical product comprises a medically acceptable yeast that is capable of fermentation.

2. The pharmaceutical product according to claim 1, wherein said yeast is selected from the group of yeasts consisting of Saccharomyces cerevisiae, Saccharomyces carlsbergensis, Saccharomyces uvarum, Saccharomyces boulardii, Saccharomyces exiguus and Saccharomyces ludwigii, Candida famala, Candida stellata, Dekkera bruxellensis, Hanensula uvarum, Kluyveromyces lactis, Kluyveromyces thermotolerans, Metschnikowia pulcherrima, Torulaspora delbrueckii and mixtures of said yeasts.

3. The pharmaceutical product according to claim 1, wherein said yeast is a yeast selected from the group consisting of a fresh yeast and a dry yeast.

4. The pharmaceutical product according to claim 1, wherein said pharmaceutical product further contains at least one carbohydrate which is/are selected from the group consisting of glucose, fructose, galactose, saccharose, maltose, maltotriose, raffinose, starch, starch derivatives and dextrins.

5. The pharmaceutical product according to claim 1, wherein said pharmaceutical products further comprises a separate compartment for containing water.

6. The pharmaceutical product according to claim 1, wherein said pharmaceutical product comprises a coating comprising a material selected from the group consisting of cellulose ethers, cellulose esters, polyacrylate, polymethacrylate, polyvinyl derivative, copolymer of polymethyl vinyl ether and malonic acid.

7. The pharmaceutical product according to claim 6, wherein the material for said coating comprises at least one excipient which is/are selected from the group consisting of plasticiser(s), wetting agent(s) and pigment(s).

8. The pharmaceutical product according to claim 7, wherein the plasticiser(s) is/are selected from the group consisting of triethyl citrate, tributyl citrate, acetyl triethyl citrate, dibutyl tartrate, diethyl sebacate, dimethyl phthalate, diethyl phthalate, dioctyl phthalate, castor oil, sesame seed oil, glyceryl triacetate, glyceryl diacetate, glycerine, 1,2-propylene glycol and polyethylene glycols.

9. The pharmaceutical product according to claim 7, wherein the wetting agent(s) is/are selected from the group consisting of PEG 400 stearate, sorbitan monooleate and PEG sorbitan monooleate.

10. The pharmaceutical product according to claim 7, wherein the pigments is selected from the group consisting of titanium dioxide and iron oxide.

11. A method for producing a pharmaceutical product for the continuous release of active pharmaceutical ingredient, comprising the steps of:
compressing an active ingredient-containing layer and a yeast-containing layer into a bilayer tablet;
providing bilayer tablet with a water-permeable gas-tight coating; and
drilling opening in the coating in the region of the active ingredient-containing layer.

12. A method for producing a pharmaceutical product for a burst release of active pharmaceutical ingredient, comprising the steps of:
incorporating an active pharmaceutical ingredient and yeast in a sugar syrup to obtain a mixture;
forming small spheres; and
providing the mixture with a gas-tight water-permeable coating after the forming of the small spheres.

13. Use of a medically acceptable yeast that is capable of alcoholic fermentation, for producing a pharmaceutical product for the controlled release of the active pharmaceutical ingredient contained in said pharmaceutical product.
